SEQUENCE LISTING (41 sequences)

SEQ ID NO: 1 - protein, 11 aa, Synthetic construct
KASQSVSNDVT

SEQ ID NO: 2 - protein, 7 aa, Synthetic construct
SASNRYS

SEQ ID NO: 3 - protein, 6 aa, Synthetic construct
QQDYSS

SEQ ID NO: 4 - protein, 5 aa, Synthetic construct
NYGVH

SEQ ID NO: 5 - protein, 16 aa, Synthetic construct
VIWAGGITNYNSAFMS

SEQ ID NO: 6 - protein, 11 aa, Synthetic construct
RGGHYGYALDY

SEQ ID NO: 7 - protein, 254 aa, Synthetic construct
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSS

SEQ ID NO: 8 - protein, 105 aa, Synthetic construct
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKR

SEQ ID NO: 9 - protein, 119 aa, Synthetic construct
QVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSS

SEQ ID NO: 10 - protein, 110 aa, Synthetic construct
QAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 11 - protein, 119 aa, Synthetic construct
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSS

SEQ ID NO: 12 - protein, 39 aa, Homo sapiens
KPLDGEYFTLQIRGRERFEMFRELNEALELKDAQAGKEP

SEQ ID NO: 13 - protein, 56 aa, Homo sapiens
RSPDDELLYLPVRGRETYEMLLKIKESLELMQYLPQHTIETYRQQQQQQHQHLLQK

SEQ ID NO: 14 - protein, 52 aa, Homo sapiens
RHGDEDTYYLQVRGRENFEILMKLKESLELMELVPQPLVDSYRQQQQLLQRP

SEQ ID NO: 15 - protein, 27 aa, Homo sapiens
QAIKKELTQIKQKVDSLLENLEKIEKE

SEQ ID NO: 16 - protein, 54 aa, Homo sapiens
STRRILGLAIESQDAGIKTITMLDEQKEQLNRIEEGLDQINKDMRETEKTLTEL

SEQ ID NO: 17 - protein, 97 aa, Homo sapiens [sequence omitted]

SEQ ID NO: 18 - protein, 30 aa, Synthetic construct
DEISMMGRVVKVEKQVQSIEHKLDLLLGFY

SEQ ID NO: 19 - protein, 60 aa, Synthetic construct
TVAEAKRQAAEDALAVINQQEDSSESCWNCGRKASETCSGCNTARYCGSFCQHKDWEKHH

SEQ ID NO: 20 - protein, 5 aa, Synthetic construct
GGGGS

SEQ ID NO: 21 - protein, 9 aa, Synthetic construct
TPLGDTTHT

SEQ ID NO: 22 - protein, 589 aa, Synthetic construct
EIVMTQTPATLSVSAGERVTITCKASQSVSNDVTWYQQKPGQAPRLLIYSASNRYSGVPARFSGSGYGTEFTFTISSVQSEDFAVYFCQQDYSSFGCGTKLEIKRGGGGSGGGGSGGGGSGGGGSGGGGSGGGGSQVQLVESGPGVVQPGRSLRISCAVSGFSVTNYGVHWVRQPPGKCLEWLGVIWAGGITNYNSAFMSRLTISKDNSKNTVYLQMNSLRAEDTAMYYCASRGGHYGYALDYWGQGTLVTVSSGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGA

SEQ ID NO: 23 - protein, 9 aa, Synthetic construct
TGAVTASNY

SEQ ID NO: 24 - skipped

SEQ ID NO: 25 - protein, 9 aa, Synthetic construct
ALWYSDHWV

SEQ ID NO: 26 - protein, 8 aa, Synthetic construct
GFSLTDYG

SEQ ID NO: 27 - protein, 7 aa, Synthetic construct
IWSGGGT

SEQ ID NO: 28 - protein, 13 aa, Synthetic construct
ARRGSYPYNYFDA

SEQ ID NO: 29 - protein, 6 aa, Synthetic construct
EDIYNR

SEQ ID NO: 30 - skipped

SEQ ID NO: 31 - protein, 9 aa, Synthetic construct
QQYWSNPYT

SEQ ID NO: 32 - protein, 8 aa, Synthetic construct
GFSLTSYG

SEQ ID NO: 33 - protein, 7 aa, Synthetic construct
MWRGGST

SEQ ID NO: 34 - protein, 13 aa, Synthetic construct
AKSMITTGFVMDS

SEQ ID NO: 35 - protein, 256 aa, Synthetic construct
QVQLQESGPGLVKPSETLSLTCTVSGFSLTSYGVHWVRQPPGKGLEWIGVMWRGGSTDYNAAFKSRVTISKDNSKNQVSLKLSSVTAADTAVYYCAKSMITTGFVMDSWGQGTLVTVSSGGGGSGGGGSGGGGSGGGGSGGGGSGGGGSDIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGQGTKLEIK

SEQ ID NO: 36 - protein, 107 aa, Synthetic construct
DIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGQGTKLEIK

SEQ ID NO: 37 - protein, 119 aa, Synthetic construct
QVQLQESGPGLVKPSETLSLTCTVSGFSLTSYGVHWVRQPPGKGLEWIGVMWRGGSTDYNAAFKSRVTISKDNSKNQVSLKLSSVTAADTAVYYCAKSMITTGFVMDSWGQGTLVTVSS

SEQ ID NO: 38 - protein, 592 aa, Synthetic construct
QVQLQESGPGLVKPSETLSLTCTVSGFSLTSYGVHWVRQPPGKGLEWIGVMWRGGSTDYNAAFKSRVTISKDNSKNQVSLKLSSVTAADTAVYYCAKSMITTGFVMDSWGQGTLVTVSSGGGGSGGGGSGGGGSGGGGSGGGGSGGGGSDIQLTQSPSSLSASVGDRVTITCKASEDIYNRLTWYQQKPGKAPKLLISGATSLETGVPSRFSGSGSGKDYTFTISSLQPEDFATYYCQQYWSNPYTFGQGTKLEIKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGQGTLVTVSSGGGGSGGGGSGGGGSGGGGSGGGGSGGGGSQAVVTQEPSLTVSPGGTVTLTCGSSTGAVTASNYANWVQQKPGQAPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAP

SEQ ID NO: 39 - protein, 591 aa, Synthetic construct
QVQLVQSGAEVKKPGASVKVSCKASGYTFTNYDINWVRQATGQGLEWMGWIFPGDGSTQYNEKFQGRVTMTTNTSISTAYMELSSLRSEDTAVYYCARQTTATWFAYWGQGTLVTVSSGGGGSGGGGSGGGGSGGGGSGGGGSGGGGSEIVMTQSPATLSVTPKEKVTITCRASQSISDYLHWYQQKPDQSPKLLIKYASQSISGVPSRFSGSGSGSDFTLTINSLEAEDAATYYCQNGHSFPLTFGQGTKLEIKGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGQGTLVTVSSGGGGSGGGGSGGGGSGGGGSGGGGSGGGGSQAVVTQEPSLTVSPGGTVTLTCGSSTGAVTASNYANWVQQKPGQAPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAP

SEQ ID NO: 40 - protein, 592 aa, Synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGQGTLVTVSSGGGGSGGGGSGGGGSGGGGSGGGGSGGGGSQAVVTQEPSLTVSPGGTVTLTCGSSTGAVTASNYANWVQQKPGQAPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGA

SEQ ID NO: 41 - protein, 591 aa, Synthetic construct
EVQLVESGGGLVKPGGSLRLSCAASGFAFSTYDMSWVRQAPGKCLEWVSTISSGGSYTYYADSVKGRFTISRDNAKNSLYLQMNSLRAEDTAVYYCAPTTVVPFAYWGQGTLVTVSAGGGGSGGGGSGGGGSGGGGSGGGGSGGGGSDIQMTQSPSSLSASVGDRVTITCKASQNVRTVVAWYQQKPGKAPKTLIYLASNRHTGVPSRFSGSGSGTEFTLTISNLQPEDFATYYCLQHWSYPLTFGCGTKLEVKRGGGGSGGGGSGGGGSGGGGSHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGTPLGDTTHTSGKPLDGEYFTLQIRGRERFEMFRELNEALELKDAQAGKEPGGSGGAP

(19) United States
(12) Patent Application Publication
(10) Pub. No.: US 2025/0041410 A1
(43) Pub. Date: Feb. 6, 2025

(54) Formulations Comprising a SADA Complex

(71) Applicant: Y-mAbs Therapeutics, Inc., New York, NY (US)

(72) Inventors: Torben LUND-HANSEN, Roskilde (DK); Nico LIEBENBERG, Fredensborg (DK); Matias Munck MORTENSEN, Roskilde (DE); Steen LISBY, Hørsholm (DK); Li PING, Værløse (DK)

(21) Appl. No.: 18/717,256

(22) PCT Filed: Dec. 14, 2022
(86) PCT No.: PCT/DK2022/050279

§ 371 Date: Jun. 6, 2024

(30) Foreign Application Priority Data
Dec. 15, 2021 (DK) PA 2021 70621

(51) Int. Cl. A61K 39/395; A61K 39/00; A61K 51/04; A61P 35/00; C07K 16/28; C07K 16/30

(52) U.S. Cl. CPC A61K 39/39558; A61K 39/3955; A61K 51/0482; A61P 35/00; C07K 16/2803; C07K 16/2827; C07K 16/2887; C07K 16/2896; C07K 16/3084; A61K 2039/505; A61K 2121/00; A61K 2123/00

(57) ABSTRACT

Disclosed are formulations comprising SADA-complex. The formulations provide for a satisfactory shelf-life without excessive disassembly or multimerization of the SADA-complex. Further disclosed is the use of the formulation for treating cancer.

The present specification comprises a sequence listing in computer readable format, submitted together with the application. The sequence listing forms part of the disclosure and is incorporated in the specification in its entirety.

The present invention relates to compositions comprising a SADA complex, wherein said SADA complex remain stabilized on tetramer-form. Preferably the composition is a pharmaceutical composition.

The invention further relates to the treatment of cancer using the composition of the invention.

### TECHNICAL BACKGROUND

Protein drugs are typically formulated as an aqueous formulation comprising ingredients that stabilizes the proteins in order to secure a satisfactory shelf-life.

The self-assembling and disassembling (SADA) technology was originally disclosed in WO 2018204873A1, and make use of SADA domains having the property of assembly and disassembly depending on concentration. Complexes comprising a SADA domain typically exists in at least two distinct forms, a tetrameric form at high concentration and a monomeric form at low concentration.

SADA-complexes may be designed so that the tetrameric form has a molecular weight well above the renal clearance limit and the monomeric form has a molecular weight below the renal clearance limit, meaning that the tetrameric form will have a high plasma-half-life and the monomeric form has a low plasma half-life.

Since SADA-complexes are mainly administered in tetrameric form it provides a challenge to the formulation thereof, because the formulation should not only provide for a satisfactory stability of the protein, it should also secure that the SADA-complex remains on tetrameric form without excessive disassembly to monomers or agglomeration to multimers.

### SUMMARY OF THE INVENTION

The present disclosure provides compositions comprising SADA domains as a part of a SADA-complex permitting effective delivery of a payload to a target site of interest, while minimizing risk of off-target interactions. For optimal delivery of a payload, it is desirable that the SADA-complex in tetramer-form is highly stable in the composition/solution. However, ensuring the stability of said compositions is a challenge. The challenge is to ensure that the composition comprises SADA-complexes predominantly in the higher-order tetramerized state, that said SADA-complexes remains on the tetramer-form and at the same time avoid multimerization, aggregation and precipitation thereof as well as product loss.

It is desirable that the SADA complex is administered on tetrameric form, because the tetrameric form having a size well above the renal clearance limit will remain in the blood circulation for a sufficient time to allow binding to the site of interest, whereas the monomeric form will be rapidly lost from the circulation because its size is below the renal clearance. In total this provides the particular desirable properties of SADA-complexes, that when administered on tetrameric form remains sufficiently long in circulation to bind to the site of interest, and complexes that do not bind a target will gradually disintegrate into monomers that will be lost via the kidneys. The present disclosure provides a composition ensuring the needed stability of the SADA-complex.

In a first aspect the invention relates to an aqueous composition comprising
a. A SADA-complex comprising a SADA domain and at least one additional domain in an amount of 5-50 g/L;
b. A buffer-system;
c. One or more stabilizing agents; and
d. One or more surfactants;
wherein the pH is in the range of 5-6, and the SADA-complex is predominantly on the tetrameric form, and the ionic strength is in the range of 5-150 mM.

It has surprisingly been realized that the formulation is capable of stabilizing the SADA-complexes upon storage and further to maintain the SADA complex predominantly on tetrameric form.

The SADA-complex preferably comprises a SADA-domain and two binding sites, one capable of binding a tumor antigen, the other binding site capable of binding a chelator complexing a metal ion. The chelator may be DOTA or a compound comprising a DOTA ring system.

In a second aspect the invention relates to the use of a composition according to the invention for treating or diagnosing cancer.

In a preferred embodiment the invention relates to the use of a composition according to the invention in a method comprising the steps of:
a. Administering a composition according to the invention to a patient in need of the treatment or diagnosing; and
b. After a period administering a DOTA-compound binding a radionuclide.

In a third aspect the invention relates to a kit comprising the composition of the invention and preferably, instructions for use and/or DOTA binding a radionuclide.

Additional aspects are provided in the claims.

### DETAILED DISCLOSURE

The present invention relates to Self Assembly and Dis-Assembly (SADA) technology that has been described in the international patent application with publication number WO 2018204873A1, incorporated herein by reference. The technology is based on SADA-domains, small polypeptides that have the property of self assembly and disassembly depending on concentration. Examples of a SADA polypeptide is a polypeptide that comprises a tetramerization domain of p53, p63, p76, hnRNPC, SNAP-23, Stefin B, KCNQ4, CBFA2T1 and any other examples of such polypeptides provided in said international patent application, without limitation.

According to the present specification, a SADA-complex is intended to mean a polypeptide comprising a SADA domain and at least one additional domain.

SADA-complexes will self-assemble and form multimers, in particular tetramers, at high concentration and disassemble into monomers at low concentration. This has the consequence that a SADA-complex on tetrameric form will, when administered to a patient, be diluted in plasma and gradually disassemble into monomers. If a SADA complex is designed so the multimeric form has a size above the renal clearance limit and the monomer has a size below the renal clearance limit, the multimer will have a long plasma half life whereas the monomer has a low plasma half life.

For SADA-complexes comprising a binding site, binding to a tissue antigen, SADA-complex will rapidly bind to the antigen target and be localized at the target tissue, whereas unbound SADA complex will rapidly disassemble and be cleared from the plasma by renal clearance.

Some embodiments of the present invention are provided in the claims.

According to an embodiment, the invention concerns an aqueous composition comprising
a. A SADA-complex, comprising a SADA domain and at least one additional domain, in an amount of 5-50 g/L;
b. A buffer-system;
c. One or more stabilizing agents; and
d. One or more surfactants;
wherein the pH is in the range of 5-6, and the ionic strength is in the range of 5-150 mM.

Preferably, the SADA complex is predominantly of multimeric/tetrameric form.

The formulation of the invention has the advantage of ensuring the stability of the composition. The inventors have realized that the formulation of the invention secures a high stability of the SADA complexes and is capable of maintaining the SADA-complexes in tetrameric form upon storage and further protect the protein against protein degradation. Thus, the compositions of the invention provide a solution of tetrameric SADA-complexes that remain on tetrameric form and reduces protein degradation after and during storage. Thus, the formulations of the invention provide SADA-molecules with a desirable high shelf-life.

According to an embodiment, the invention concerns the composition of the invention, wherein the ionic strength is in the range of 5-150 mM, 10-135 mM, 20-120 mM or 25-100 mM.

According to an embodiment, the invention concerns the composition of the invention, comprising a SADA-complex in an amount selected among 5-50 g/L, 6.25-45 g/L, 7.5-40 g/L, 9.75-35 g/L, 10-20 g/L, and preferably 10-15 g/L.

According to an embodiment, the SADA-complex comprises two binding sites and a SADA domain, wherein the first binding site is capable of binding to a target antigen and the second binding site is capable of binding to a payload, such as a cytotoxic agent, a radionuclide or a compound capable of binding a payload.

In some embodiments the first and/or second binding site is or comprises an antibody component, such as an antigen binding fragment of an antibody, a scFv or a nanobody. Preferably, the first and/or second binding sites is (are) a scFv.

In some embodiments the first binding site is specific for a cell surface target, such as a tumor antigen.

According to an embodiment the binding site specific for a tumor antigen is anti-GD2, anti-CD20, anti-CD38, anti-Globo H, anti-GPA33, anti-PSMA, anti-polysialic acid, anti-Lewy, anti-LiCAM, anti-HER2, anti-B7H3, anti-CD33, anti-peptide/MHC, anti-glypican3, or anti GD3 binding domain.

Accordingly, the invention concerns the composition of the invention, wherein the first binding site is capable of binding to a tumor antigen.

According to an embodiment, the invention concerns the composition according to the invention, wherein the first binding site is capable of binding to GD2, B7-H3, CD20, GPA33 or CD38.

GD2 is a disialoganglioside, which can be considered a tumor-associated antigen.

B7-H3 also known as CD276 is an immune checkpoint molecule and a costimulatory/coinhibitory immunoregulatory protein, which can be considered a tumor-associated antigen.

CD20 is a membrane-embedded surface molecule, which can be considered a tumor-associated antigen.

GPA33 is a glycoprotein and a cell surface antigen, which can be considered a tumor-associated antigen.

CD38, also known as cyclic ADP ribose hydrolase, is a glycoprotein, which can be considered a tumor-associated antigen.

According to an embodiment, the invention concerns the composition according to the invention, wherein the first binding site comprises a sequence
a. Comprising the CDR sequences of SEQ ID NO: 1-6 and
b. Having at least 90%, 95%, 96%, 97% 98% or preferably at least 95% sequence identity, to SEQ ID NO: 7.

According to one preferred embodiment the first binding site is capable of binding GD2 and comprises the sequence of SEQ ID NO: 7.

According to an embodiment, the invention concerns the composition according to the invention, wherein the first binding site comprises a sequence
a. Having at least 90%, 95%, 96%, 97% 98% or preferably at least 95% sequence identity, to SEQ ID NO: 8; and a sequence
b. Having at least 90%, 95%, 96%, 97% 98% or preferably at least 95% sequence identity, to SEQ ID NO: 9.

CD38, also known as cyclic ADP ribose hydrolase, is a glycoprotein, which can be considered a tumor-associated antigen.

According to an embodiment, the invention concerns the composition according to the invention, wherein the first binding site comprises a sequence
a. Comprising the CDR sequences of SEQ ID NO: 29-34 and
b. Having at least 90%, 95%, 96%, 97% 98% or preferably at least 95% sequence identity, to SEQ ID NO: 35.

Preferably, the first binding site of this embodiment is capable of binding CD38.

In a preferred embodiment, the first binding site is capable of binding CD38 and comprises the sequence of SEQ ID NO: 35.

According to another embodiment, the invention concerns the composition according to the invention, wherein the first binding site comprises a sequence
a. Having at least 90%, 95%, 96%, 97% 98% or preferably at least 95% sequence identity, to SEQ ID NO: 36 and a sequence
b. Having at least 90%, 95%, 96%, 97% 98% or preferably at least 95% sequence identity, to SEQ ID NO: 37.

According to an embodiment, the invention concerns the composition according to the invention, wherein the second binding site is capable of binding to a chelator.

In principle any chelator may be used according to the invention, provided that the second binding site is capable of binding said chelator.

According to an embodiment, the invention concerns the composition according to the invention, wherein the second binding site is capable of binding to DOTA, or a compound comprising a DOTA ring system, or capable of binding DOTA or a compound comprising a DOTA ring system when DOTA is chelated to a metal ion, e.g. lutetium such as ¹⁷⁵Lu³⁺ or ¹⁷⁷Lu³⁺.

DOTA (Dodecane Tetraacetic Acid) is also referred to as 1,4,7,10-tetraazacyclododecane-1,4,7 10-tetraacetic acid, and has the formula (CH₂CH₂NCH₂CO₂H)₄ also known as C₁₆H₂₈N₄O₈·xH₂O.

A compound comprising a DOTA ring system is in this specification intended to mean a compound comprising DOTA whereto additional groups or moieties are attached. Examples of such compounds include Benzyl-DOTA and the bispecific chelators disclosed in WO2019010299A, incorporated by reference.

According to an embodiment, the invention concerns the composition according to the invention, wherein the second binding site:
a. Comprises the CDR sequences of SEQ ID NO: 23-28, and
b. Comprising a polypeptide with at least 90%, 95%, 96%, 97% 98% or preferably at least 95% sequence identity, to SEQ ID NO: 35.

Preferably, the second binding site of this embodiment is capable of binding DOTA-metal, i.e. DOTA chelating a metal ion such as lutetium, preferably Lu³⁺.

According to this embodiment, the invention concerns the composition according to the invention, wherein the second binding site is capable of binding DOTA-metal and comprises a sequence
a. with at least 90%, 95%, 96%, 97% 98% or preferably at least 95% sequence identity, to SEQ ID NO: 10 and a sequence
b. with at least 90%, 95%, 96%, 97% 98% or preferably at least 95% sequence identity, to SEQ ID NO: 11.

According to an embodiment, the invention concerns the composition according to the invention, wherein the SADA-domain comprises a sequence disclosed in SEQ ID No. 12-19 or a sequence that differs from one of the sequences SEQ ID NO: 12-19 by 1, 2, 3, 4, 5, 6, 7, 8, 9 or 10 substitutions.

The p53 tetramerization domain comprising the sequence of SEQ ID NO: 12, more preferred amino acids 6-36 of SEQ ID NO: 12, is a preferred SADA domain.

The SADA-complexes may according to the invention comprise additional elements, including but not limited to linkers separating different parts of the complex, antibody fragments apart from binding sites, such as constant regions and antigen fragments binding to and capable of eliciting effector or immune reactions.

According to an embodiment, the SADA complex comprises linkers.

Linkers also sometimes known as spacers are short amino acid sequences created to separate two domains in a single polypeptide, allowing the two domains to fold and operate without steric hindrance from an adjacent domain. Linkers are known in the art and the present invention is not limited to any particular sequence of the linkers. In general, the purpose of linkers is to connect and/or separate different elements of the complex and are typically mainly composed of small hydrophilic amino acids such as glycine, serin and threonine.

According to an embodiment, the invention concerns the composition according to the invention, wherein the SADA complex comprises linkers with a sequence selected among SEQ ID NO: 20 multiplied by an integer between 1-6.

Another suitable linker that may be used according to the invention is an IgG3 spacer domain, such as the IgG3 spacer domain is disclosed in SEQ ID NO: 21.

In some embodiments the SADA-complex consists of a SADA domain and 2 binding sites such as scFv's. In some embodiments the SADA-complex comprises anti-GD2 scFv-anti-DOTA scFv-p53 tetramerization domain connected by linkers and/or spacers. In some embodiments the SADA-complex has the following structure: anti-GD2 light chain Fv-anti-GD2 heavy chain Fv-anti-DOTA heavy chain Fv-anti-DOTA light chain Fv-p53 tetramerization domain connected by linkers and/or spacers.

Examples of suitable SADA complexes according to the invention includes the GD2-SADA conjugate comprising the amino acid sequence of SEQ ID NO: 22, the CD38-SADA conjugate comprising the amino acid sequence of SEQ ID NO: 38, the B7-H3-SADA conjugate comprising the amino acid sequence of SEQ ID NO: 39, the CD20-SADA conjugate comprising the amino acid sequence of SEQ ID NO: 40 and the GPA33-SADA conjugate comprising the amino acid sequence of SEQ ID NO: 41.

According to the invention, the composition according to the invention, comprises a buffer system such as an organic acid or an alkali metal salt thereof.

Preferably, the buffer is selected among acetate, citrate, histidine, citrate-histidine, acetate-histidine and succinate.

Preferred examples include acetate buffer, comprising acetic acid and sodium acetate.

Sodium acetate is also known as Acetic acid sodium salt and has the Formula CH₃COONa.

According to an embodiment, the invention concerns the composition according to the invention, comprising a buffer in an amount selected among 5-30 mM, 10-25 mM and preferably 20 mM.

According to an embodiment, the stabilizing agent is selected among polyols, in particular sugar alcohols and non-reducing sugars.

Preferred examples include sucrose, trehalose, sorbitol, glycerol and inositol.

The stabilizer maintains or extends the time, wherein the active pharmaceutical ingredient maintains the desirable properties during storage.

According to an embodiment, the invention concerns the composition according to the invention comprising a stabilizing agent, preferably sucrose, in an amount selected among, 200-600 mM, 225-500 mM, 250-300 mM, and preferably 275 mM.

The invention also concerns a composition, wherein the surfactant is a nonionic surfactant.

Nonionic surfactants may comprise/consist of long chain polymers which do not dissociate, consisting of a hydrophilic head group and a hydrophobic tail.

According to an embodiment, the invention concerns a composition wherein the surfactant is Polyethylene glycol sorbitan monolaurate, Poly(ethylene glycol)-block-poly(propylene glycol)-block-poly(ethylene glycol) or Polyethylene glycol sorbitan monooleate, Polyoxyethylenesorbitan monooleate.

Polyethylene glycol sorbitan monolaurate, also known as polyoxyethylenesorbitan monolaurate, is known in the art. A preferred Polyethylene glycol sorbitan monolaurate is commercially available as Polysorbate® 20 or TWEEN® 20.

Polyethylene glycol sorbitan monooleate, also known as polyoxyethylenesorbitan monooleate, is also known in the art. A preferred Polyethylene glycol sorbitan monooleate is commercially available as Polysorbate® 80 or TWEEN® 80.

Poly(ethylene glycol)-block-poly(propylene glycol)-block-poly(ethylene glycol) is also known in the art. A preferred Poly(ethylene glycol)-block-poly(propylene glycol)-block-poly(ethylene glycol) is commercially available as Kolliphor® P188 or Poloxamer® 188.

Polyethylene glycol sorbitan monolaurate is a preferred surfactant for use according to the invention.

According to an embodiment, the invention concerns compositions, comprising a surfactant in an amount selected among 0.1-0.3 g/L, 0.15-0.25 g/l, 0.16-0.24 g/L, 0.17-0.23 g/L, 0.18-0.22 g/L, 0.19-0.21 g/L and preferably 0.20 g/L.

The compositions of the invention may have a pH selected among 5-6, 5.1-5.9, 5.2-5.8, 5.3-5.7, 5.4-5.6 and preferably 5.5.

The compositions according to the invention may further comprise an antioxidant.

An antioxidant can be added to a composition to protect the contents from damage caused by oxidative stress. This can be advantageous for proteins comprising amino acids susceptible to oxidation particularly for proteins comprising amino acids susceptible of oxidation which amino acids are exposed on the surface of the proteins. Example of amino acids susceptible to oxidation includes residues such as methionine and (free) cysteine.

A preferred antioxidant for use according to the invention is Methionine.

According to an embodiment, the invention concerns compositions, comprising an antioxidant, such as methionine, in an amount selected among, 5-15 mM, 6-14 mM, 7-13 mM, 8-12 mM, 9-11 mM and preferably 10 mM.

The invention further concerns a composition according to the invention, that does not comprise salt, or only comprise salt in a low concentration e.g. below 50 mM.

The inventors have realized that salts in general destabilizes SADA complexes, and it is therefore preferred to limit the amounts of salts such as NaCl, KCl, or similar salts; in the composition.

A preferred composition according to the invention, comprises
a. SADA-complex in an amount of 15 g/L
b. Sodium acetate in an amount of 20 mM
c. Sucrose in an amount of 275 mM
d. Polysorbate 20 in an amount of 0.2 g/L
wherein the pH is 5,5 and the SADA complex is predominantly on tetrameric form.

Optionally, the composition further comprises 10 mM methionine.

The term predominantly on tetramer-form is in the present specification and claims intended to mean that the majority of the SADA-complexes are on tetramer-form, e.g., at least 50% w/w; at least 60% w/w; at least 70% w/w; at least 80% w/w; at least 90% w/w; or at least 95% w/w.

According to an embodiment, the composition according to the invention is a pharmaceutical composition.

The SADA-complexes of the invention may be prepared using methods known in the art.

In a preferred embodiment, a nucleic acid encoding the desired amino acid sequence of the complex is provided. A construct comprising the nucleic acid sequence provided with the necessary regulatory elements to direct expression in a selected host organism, such as promoter, signal sequence ribosome recognition sites Kozak sequence, enhancers, terminator, poly adenylation sites etc. is prepared and inserted into the selected host organism that is grown under conditions leading the expression of the SADA-complex.

Finally, the SADA-complex is recovered from the growth broth using well known separation and recovery techniques.

The formulation of the invention may be prepared by dissolving the SADA-complex and other ingredients in sterile water using method known in the art.

The invention further concerns use of a composition according to the invention for treating or diagnosing cancer.

According to an embodiment, the composition according to the invention may be used for treating or diagnosing cancer expressing the tumor antigen recognized by the SADA conjugate, such as GD2, CD38, B7-H3, CD20 or GPA33.

The cancer may be selected among neuroblastoma, melanoma, sarcoma, brain tumor or carcinoma.

According to an embodiment, the invention concerns use of a composition according to the invention, wherein said cancer is selected among osteosarcoma, liposarcoma, fibrosarcoma, malignant fibrous histiocytoma, leiomyosarcoma, spindle cell sarcoma, brain tumor, small cell lung cancer, retinoblastoma, HTLV-1 infected T cell leukemia and other tumors that are positive for GD2, CD38, B7-H3, CD20 or GPA33.

According to an embodiment, the invention concerns use of a composition according to any of the preceding claims, in a method comprising the steps:
a. Administering a composition according to the invention to a patient in need of the treatment or diagnosing; and
b. After a period administering a DOTA-compound comprising a radionuclide.

The period in step b. is typically selected between 48 hours to 72 hours, such as 50 hours to 65 hours, or 55 hours to 60 hours. Preferably, the period is selected to allow the majority of unbound SADA-complex to disassemble and be cleared from the blood stream.

The method of the invention may further comprise administering a clearing agent after step a. and before step b.

According to an embodiment, the invention concerns use of a composition according to the invention, wherein the radionuclide is selected among an alpha, beta and positron emitting radionuclide.

According to an embodiment, the invention concerns use according to the invention, wherein the radionuclide is selected from the group consisting of ²¹¹At, ⁵¹Cr, ⁵⁷Co, ⁵⁸Co, ⁶⁷Cu, ¹⁵²Eu, ⁶⁷Ga, ¹¹¹In, ⁵⁹Fe, ²¹²Pb, ¹⁷⁷Lu, ²²³Ra, ²²⁴Ra, ¹⁸⁶Re, ¹⁸⁸Re, ⁷⁵Se, ^{99m}Tc, ²²⁷Th, ⁸⁹Zr, ⁹⁰Y, ^{94m}Tc, ⁶⁴Cu, ⁶⁸Ga, ⁶⁶Ga, ⁸⁶Y, ⁸²Rb, ^{110m}In, ²⁰⁹Bi, ²¹¹Bi, ²¹²Bi, ²¹³Bi, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf.

Preferred examples of radionuclides for use according to the invention includes ¹⁷⁷Lu, ^{99m}Tc, ⁶⁴Cu, ⁹⁰Y and ⁸⁹Zr.

According to an embodiment, the invention concerns kit comprising the composition of the invention, and a DOTA compound.

Typically, the kit further comprises instructions to use, or at least information to the user regarding where to find such information.

According to an embodiment, the invention concerns kit according to the invention, further comprising a radionuclide.

### Definitions

Antibody fragment: An antibody fragment is a portion of an antibody such as F(ab′)2, F(ab)2, Fab′, Fab, Fv, sFv and the like. Regardless of structure, an antibody fragment binds with the same antigen that is recognized by the intact antibody. For example, an 3F8 monoclonal antibody fragment binds with an epitope recognized by 3F8. The term “antibody fragment” also includes any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.

DOTA: DOTA (Dodecane Tetraacetic Acid) is also referred to as 1,4,7,10-tetraazacyclododecane-1,4,7 10-tetraacetic acid, and has the formula (CH₂CH₂NCH₂CO₂H)₄ also known as C₁₆H₂₈N₄O₈·xH₂O.

DOTA metal chelate: means DOTA with a complex bound metal ion.

Derivative of DOTA: is intended to mean a compound comprising the DOTA ring system and is capable of chelating metal ions. Examples of such compounds include Benzyl-DOTA and the bispecific chelators disclosed in WO2019010299A. Additional DOTA derivatives are disclosed in WO2010099536 A1.

Radioactive isotope: Examples of radioactive isotopes that can be conjugated to antibodies for use diagnostically or therapeutically include, but are not limited to, ²¹¹At, ¹⁴C, ⁵¹Cr, ⁵⁷Co, ⁵⁸Co, ⁶⁷Cu, ¹⁵²Eu, ⁶⁷Ga, ³H, ¹¹¹In, ⁵⁹Fe, ¹⁷⁷Lu, ³²p, ²²³Ra, ²²⁴Ra, ¹⁸⁶Re, ¹⁸⁸Re, ⁷⁵Se, ³⁵S, ^{99m}Tc, ²²⁷Th, ⁸⁹Zr, ⁹⁰Y, ¹²³I, ¹²⁴I, ¹²⁵I, ¹³¹I, ^{94m}Tc, ⁶⁴Cu, ⁶⁸Ga, ⁶⁶Ga, ⁷⁶Br, ⁸⁶Y, ⁸²Rb, ^{110m}In, ¹³N, ¹¹C, ¹⁸F and alpha- emitting particles. Non-limiting examples of alpha-emitting particles include ²⁰⁹Bi, ²¹¹Bi, ²¹²Bi, ²¹³Bi, ²¹²Pb, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²¹⁸Rn, ²¹⁹Rn, ²²⁰Rn, ²²²Rn, ²²⁶Rn, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf.

Treatment: As used herein, the terms “treatment”, “treat”, “treated” or “treating” refer to prophylaxis and/or therapy, particularly wherein the object is to prevent or slow down (lessen) an undesired physiological change or disorder, such as the progression of multiple sclerosis. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

Pharmaceutical composition: As used herein the term “Pharmaceutical composition” is intended to mean a composition for administration as a drug or medicine to a patient in need thereof. Pharmaceutical compositions are prepared from pharmaceutical grade ingredients e.g., as described in European Pharmacopoeia 10^{th} Edition, using methods and technologies known in the pharmaceutical or apothecary area.

Sequence identity: The term Sequence identity is intended to mean a measurement of the relatedness of two nucleic or amino acid sequences. Sequence identity is determined by aligning the two sequences and finding the longest overlap, counting the number of matches in the overlap and calculating the sequence identity by dividing the number of matches by the number of, nucleotide or amino acid, residues in the overlap. Sequence identity is typically expressed in percent (%).

A variety of computational algorithms are available for the skilled person, for generating sequence alignment and calculating Sequence identity. As used herein, Sequence alignment refers to Pairwise alignments. Several algorithms perform this including the sequence alignment program Clustal Omega [doi: 10.1038/msb.2011.75].

As used herein the sequence alignment are performed using the algorithm:
Algorithm: Clustal Omega (1.2.4),
(http://www.clustal.org/omega/).

### FIGURES

FIG. 1 shows the results of two mass photometry measurements of GD2-SADA construct at a concentration of 100 nM and 5 nM. For further details see example 1.

FIG. 2 shows the distribution of monomer, dimer and tetramer of a GD2-SADA construct dependent of the concentration. For further details see example 1.

FIG. 3 shows the design of part A, phase I trial for GD2-SADA. For further details see example 10.

FIG. 4 shows the effect of tetramerization on the tumor killing effect for a GD2-SADA conjugate. For further details see example 9.

FIG. 5 shows SPECT images of tumor implanted IMR-32 athymic nude mice that had been given GD2-SADA (>90% tetramer, 10 mg/kg, IV) (top panel), GD2-monomer (2.4 mg/kg, IV) (middle panel) and a GD2-monomer (9.6 mg/kg) (lower panel); followed by administration of ¹⁷⁷Lu-DOTA. In each panel, the first mouse was scanned 2 hours after administration of ¹⁷⁷Lu-DOTA, the following mice were scanned 24, 48 and 120 hours after administration of ¹⁷⁷Lu-DOTA, respectively. For further details see example 9.

### SEQUENCES

SEQ ID NO: 1-6: CDR sequences of the GD2 binding antibody 3F8 equal to the CDR sequences of the GD2-scFv;
SEQ ID NO: 7: anti-GD2 scFv;
SEQ ID NO 8: anti-GD2 VL
SEQ ID NO 9: anti-GD2 VH
SEQ ID NO: 10: huC825 VL;
SEQ ID NO: 11: huC825 VH
SEQ ID NO: 12-19: SADA domains disclosed in WO 2018204873A1;
SEQ ID NO: 20: Linker sequence;
SEQ ID NO: 21: IgG3 spacer sequence;
SEQ ID NO: 22: GD2-SADA complex.
SEQ ID NO: 23-28 CDR sequences of C825
SEQ ID NO: 29-34: CDR sequences of a CD38 binding antibody
SEQ ID NO: 35: anti-CD38 scFV
SEQ ID NO: 36: anti-CD38 VL
SEQ ID NO: 37: anti-CD38 VH
SEQ ID NO: 38: CD38-SADA complex
SEQ ID NO: 39: B7-H3-SADA complex
SEQ ID NO: 40: CD20-SADA complex
SEQ ID NO: 41: GPA33-SADA complex.

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 10.0.

### Methods and Materials

GD2-SADA complex: the complex comprises an anti-GD2 scFv domain, a humanized C825 domain and a P53 SADA domain. The complex is disclosed in WO 2018/204873A1 as SEQ ID NO: 31, and as SEQ ID NO: 22 in the present patent application.
CD38-SADA complex: the complex comprises an anti-CD38 scFv domain, a humanized C825 domain and a P53 SADA domain. The complex is disclosed in (unpublished Danish Patent application) DK PA 2021 70621 as SEQ ID NO:41, and as SEQ ID NO: 38 in the present patent application.
B7-H3-SADA complex: the complex comprises an anti-B7-H3 scFv domain, a humanized C825 domain and a P53 SADA domain. The complex has the amino acid sequence shown in SEQ ID NO: 39.
CD20-SADA complex: the complex comprises an anti-CD20 scFv domain, a humanized C825 domain and a P53 SADA domain. The complex is disclosed in (unpublished Danish Patent application) DK PA 2021 70621 as SEQ ID NO:42, and as SEQ ID NO: 40 in the present patent application.
GPA33-SADA complex: the complex comprises an anti-GPA33 scFv domain, a humanized C825 domain and a P53 SADA domain. The complex is disclosed in (unpublished Danish Patent application) DK PA 2021 70621 as SEQ ID NO:61, and as SEQ ID NO: 41 in the present patent application.

### Surfactants

Polysorbate 20: TWEEN 20®, Polyethylene glycol sorbitan monolaurate, Polyoxyethylenesorbitan monolaurate.
Polysorbate 80: TWEEN 80®, Polyethylene glycol sorbitan monooleate, Polyoxyethylenesorbitan monooleate. Poloxamer 188: Poly(ethylene glycol)-block-poly(propylene glycol)-block-poly(ethylene glycol)
Kollifor: Poly(ethylene glycol)-block-poly(propylene glycol)-block-poly(ethylene glycol)

### EXAMPLES

### Example 1. Impact of SADA Concentration on Relative Numbers of Oligomeric Forms Measured for GD2-SADA

For this example, a Refeyn Mass photometer (Refeyn Ltd, Oxford UK) was used according to the manufacturer's instructions, with the following settings:

| Measurement principle | Label-free/interferometric light scattering | |
|---|---|---|
| Mass range | 4 kDa-5 MDa | |
| Measurement precision | +/−2% | |
| Single measurement mass error | +/−5% | |
| Resolution (FWHM) | 25 kDa@66 kDa, 88 kDa@660 kDa | |
| Concentration range | 100 pM-100 nM (particle concentration) | |
| Sensitivity | <1 ng of protein | |
| Sample volume | 5-20 | μl |
| Frame rate/standard setting) | 1 kHz (raw), 100 Hz (integrated) | |
| Field of view | 3 × 10 μm (@ 1 kHz) up to | |
| | 10 × 10 μm (@300 Hz) | |
| Wavelength | 525 | nm |
| Pixel size | 21 | nm |

The GD2-SADA construct was diluted in PBS, and incubated for 60 minutes at 37° C. before measuring the size distribution. Following dilutions were made: 150 nM, 100 nM, 50 nM, 10 nM, 5 nM, 1 nM and 100 PM and subjected to Mass photometry using the Refeyn Mass photometer.

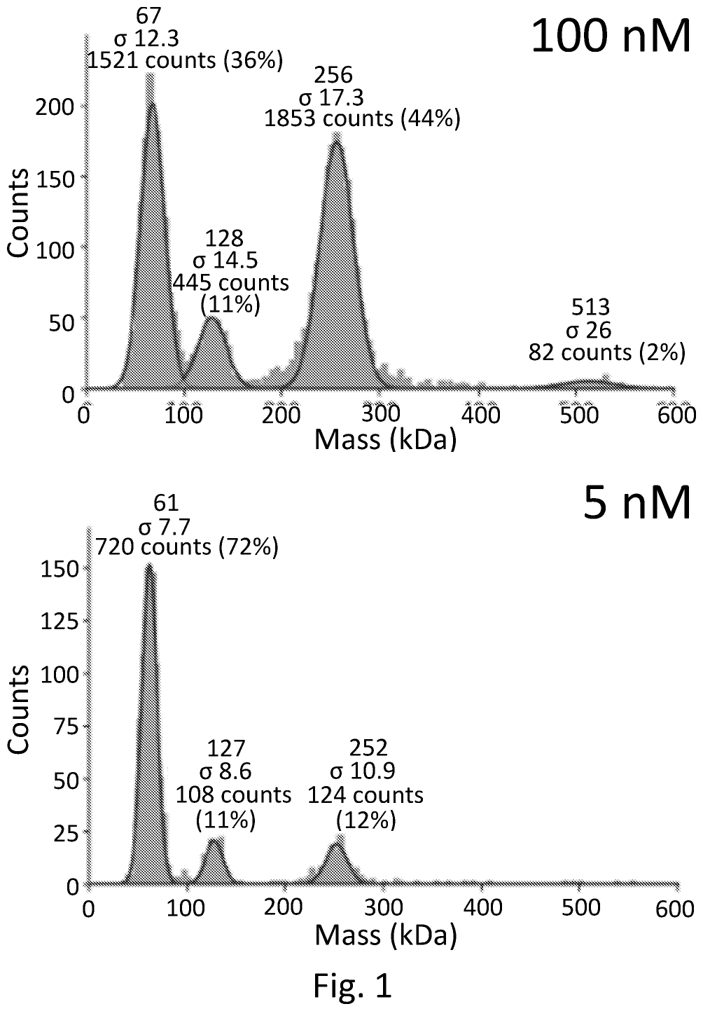

Exemplary spectrograms are shown in FIG. 1, on top for 100 nM and below for 5 nM. In the spectrograms three peaks representing monomer, dimer and tetramer GD2-SADA can be seen and for the 100 nM concentration further a broad peak at around 500 kDa can be seen representing higher multimers of GD2-SADA. It can further be seen that at high concentration (100 nM) a high proportion of the GD2-SADA constructs are present at tetrameric form, whereas at low concentration the majority is present a monomeric form.

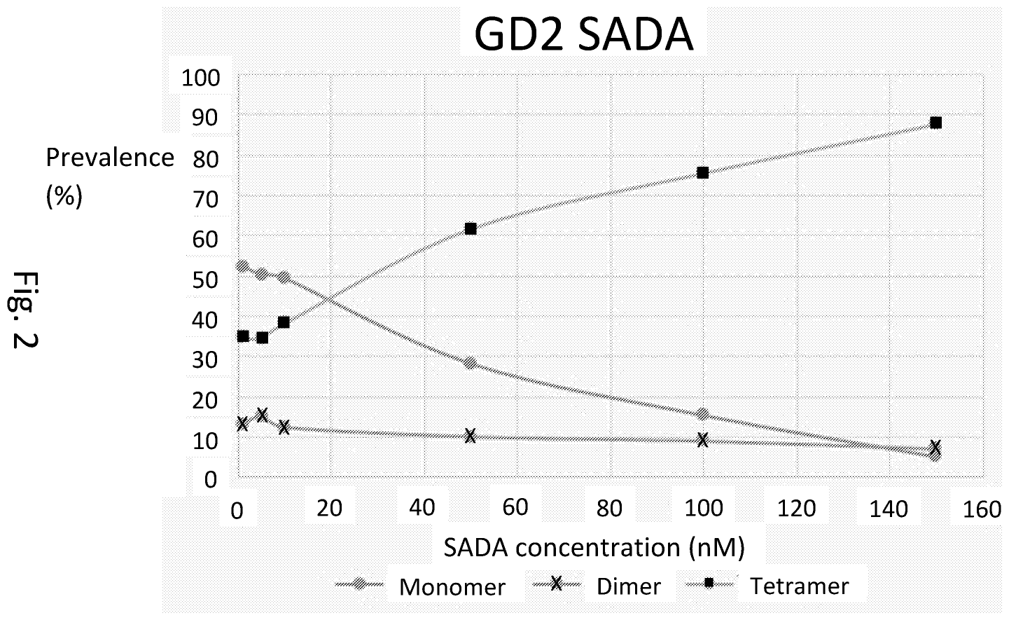

FIG. 2 shows the distribution of GD2-SADA construct dependent on concentration.

### Example 2. Effect of Buffers and pH

GD2-SADA was prepared in 20 mM buffers at the pH indicated in the table below. The stability of the construct was determined by nanoDSF, where the Tm indicated the temperature where 50% of the protein is unfolding, meaning that a higher temperature is indicative for a higher stability. The measurements were made in quadruples. Following result were obtained:

TABLE 1
GD2-SADA; Effect of buffer and pH

| | | Tm (° C.) | |
|---|---|---|---|
| Buffer | pH | Mean | SD |
| Acetate | 4.5 | 60.5 | 0.9 |
| | 5.0 | 60.7 | 0.2 |
| | 5.5 | 61.7 | 1.3 |
| Citrate | 4.5 | 58.4 | 0.3 |
| | 5.0 | 60.7 | 1.2 |
| | 5.5 | 60.4 | 0.2 |
| Histidine | 5.5 | 60.8 | 0.9 |
| | 6.0 | 62.3 | 0.4 |
| | 6.5 | 62.3 | 0.5 |
| Citrate-histidine | 5.5 | 61.9 | 0.3 |
| | 6.0 | 61.5 | 0.2 |
| | 6.5 | 61.7 | 0.2 |
| Acetate-histidine | 5.5 | 61.9 | 0.3 |
| | 6.0 | 61.5 | 0.2 |
| | 6.5 | 61.7 | 0.2 |
| Succinate | 4.5 | 59.7 | 0.6 |
| | 5.0 | 60.9 | 0.3 |
| | 5.5 | 61.2 | 0.4 |

The results showed that acetate and histidine provided a higher stability than citrate and succinate buffers. A pH value >5.5 was found to provide best stability.

### Example 3. Effect of Salt and Sucrose

GD2-SADA was prepared in 20 mM buffers at pH 5.5 and salt (sodium chloride) or sucrose added as indicated. The stability of the construct was determined by nanoDSF, where the Tm indicated the temperature where 50% of the protein is unfolding, meaning that a higher temperature is indicative for a higher stability. The measurements were made in quadruples. Following result were obtained:

TABLE 2
GD2-SADA: nanoDSF results: effect of salt and sucrose

| Buffer (20 mM, | NaCl | Sucrose | Tm (° C.) | |
|---|---|---|---|---|
| pH 5.5) | (mM) | (mM) | Mean | SD |
| Acetate | 150 | 0 | 60.9 | 0.2 |
| Citrate | 150 | 0 | 60.4 | 0.5 |
| Histidine | 150 | 0 | 60.7 | 0.7 |
| Citrate-histidine | 150 | 0 | 60.7 | 0.6 |
| Acetate- | 150 | 0 | 60.6 | 0.7 |
| histidine | | | | |
| Succinate | 150 | 0 | 60.8 | 0.2 |
| Acetate | 0 | 275 | 62.7 | 0.5 |
| Citrate | 0 | 275 | 61.4 | 0.3 |
| Histidine | 0 | 275 | 62.7 | 0.3 |
| Citrate-histidine | 0 | 275 | 61.1 | 0.0 |
| Acetate- | 0 | 275 | 63.8 | 0.3 |
| histidine | | | | |
| Succinate | 0 | 275 | 62.5 | 0.4 |

The results showed that sucrose is stabilizing the construct whereas salt (sodium chloride) is destabilizing.

### Example 4. Effect of Surfactants in an Accelerated Aging Study

Exemplary formulations were prepared, each comprising 20 mM acetate buffer and 275 mM sucrose and further comprising:

TABLE 3
GD2-SADA: Formulations for accelerated aging study

| | | | GD2-SADA |
|---|---|---|---|
| # | pH | Surfactant | concentration (mg/ml) |
| 1 | 5.5 | 0.2 g/l Polysorbate 20 | 15 |
| 2 | 5.5 | 3.0 g/l Polysorbate 20 | 15 |
| 3 | 5.5 | 0.2 g/l Polysorbate 80 | 15 |
| 4 | 5.5 | 3.0 g/l Polysorbate 80 | 15 |
| 5 | 5.0 | 0.2 g/l Poloxamer 188 | 15 |
| 6 | 5.5 | 0.2 g/l Poloxamer 188 | 15 |
| 7 | 6.0 | 0.2 g/l Poloxamer 188 | 15 |
| 8 | 5.5 | 3.0 g/l Poloxamer 188 | 15 |
| 9 | 5.5 | 0.2 g/l Poloxamer 188 | 10 |
| 10 | 5.5 | 3.0 g/l Poloxamer 188 | 10 |
| 11 | 5.5 | 0 | 15 |
| 12 | 5.5 | 0 | 10 |

The solutions were stored for one or two weeks at 40° C. and were thereafter analyzed for purity by size exclusion HPLC, and the purity drop for the main peak and the main recovery based on the recovery before incubation were calculated. The results are shown in the tables 4 and 5 below.

TABLE 4
GD2-SADA: Accelerated storage data measured by
SE-HPLC after 1 week @ 40° C.

| | | | | | | | Mean |
|---|---|---|---|---|---|---|---|
| | Conc. | | Polysorbate | Polysorbate | Poloxamer | Purity | recovery |
| # | (mg/ml) | pH | 20 (g/l) | 80 (g/l) | 188 (g(l) | drop | (%) |
| 1 | 15 | 5.5 | 0.2 | 0 | 0 | 3.4 | 96.5 |
| 2 | 15 | 5.5 | 3.0 | 0 | 0 | 3.4 | 92.5 |
| 3 | 15 | 5.5 | 0 | 0.2 | 0 | 3.5 | 96.0 |
| 4 | 15 | 5.5 | 0 | 3.0 | 0 | 3.5 | 97.3 |
| 5 | 15 | 5.0 | 0 | 0 | 0.2 | 4.3 | 85.3 |
| 6 | 15 | 5.5 | 0 | 0 | 0.2 | 3.5 | 81.7 |
| 7 | 15 | 6.0 | 0 | 0 | 0.2 | 3.1 | 82.5 |
| 8 | 15 | 5.5 | 0 | 0 | 3.0 | 3.3 | 90.9 |
| 9 | 10 | 5.5 | 0 | 0 | 0.2 | 1.8 | 86.2 |
| 10 | 10 | 5.5 | 0 | 0 | 3.0 | 2.0 | 95.6 |
| 11 | 15 | 5.5 | 0 | 0 | 0 | 3.8 | 77.7 |
| 12 | 10 | 5.5 | 0 | 0 | 0 | 2.4 | 82.2 |

TABLE 5
GD2-SADA: Accelerated storage data measured by SE-
HPLC after 2 weeks @ 40° C.

| | | | | | | | Mean |
|---|---|---|---|---|---|---|---|
| | Conc. | | Polysorbate | Polysorbate | Poloxamer | Purity | recovery |
| # | (mg/ml) | pH | 20 (g/l) | 80 (g/l) | 188 (g(l) | drop | (%) |
| 1 | 15 | 5.5 | 0.2 | 0 | 0 | 4.9 | 98.6 |
| 2 | 15 | 5.5 | 3.0 | 0 | 0 | 4.9 | 92.2 |
| 3 | 15 | 5.5 | 0 | 0.2 | 0 | 5.3 | 97.3 |
| 4 | 15 | 5.5 | 0 | 3.0 | 0 | 5.2 | 97.5 |
| 5 | 15 | 5.0 | 0 | 0 | 0.2 | 7.6 | 83.4 |
| 6 | 15 | 5.5 | 0 | 0 | 0.2 | 5.4 | 86.1 |
| 7 | 15 | 6.0 | 0 | 0 | 0.2 | 5.6 | 79.2 |
| 8 | 15 | 5.5 | 0 | 0 | 3.0 | 5.4 | 87.2 |
| 9 | 10 | 5.5 | 0 | 0 | 0.2 | 2.4 | 81.1 |
| 10 | 10 | 5.5 | 0 | 0 | 3.0 | 2.6 | 96.5 |
| 11 | 15 | 5.5 | 0 | 0 | 0 | 5.2 | 73.8 |
| 12 | 10 | 5.5 | 0 | 0 | 0 | 3.0 | 77.4 |

The results showed that surfactants improved mean recovery. In this example a better recovery was obtained using Polysorbate 20/80 or high concentration of Poloxamer 188.

### Example 5: GD2-SADA Formulation

An exemplary formulation of GD2-SADA was made taking advantage of the conclusions in examples 1-4. Further methionine was added as an antioxidant to protect M199 and Polysorbate from oxidation.

TABLE 6
GD2-SADA formulation

| Component | Amount | | Function |
|---|---|---|---|
| GD2-SADA | 15 | g/l | API |
| Sodium Acetate | 20 | mM | Buffer |
| Sucrose | 275 | mM | Stabilizing agent |
| Polysorbate 20 | 0.2 | g/l | Surfactant |
| Methionine | 10 | mM | Antioxidant |

| | |
|---|---|
| pH | 5.5 |

### Example 6: Compatibility of GD2-SADA Formulation

To demonstrate the compatibility of the formulation during clinic administration, an in use stability study was performed to demonstrate suitable product recovery and stability for the GD2-SADA formulation according to example 6 for 4 hours at room temperature including administration time.

The study covered a concentration range from 0.05 mg/ml to 10 mg/mL. Dilution of GD2-SADA was done in normal saline (NaCl 0.9%). The 50 mL IV-bag containing GD2-SADA dilutions were connected to an infusion set and infusion filter. Samples were taken by allowing GD2-SADA dilutions to pass from the 50 mL IV-bag through the infusion set and filter.

Purity, potency, physicochemical, and particle results were all within the expected range and comparable between T=0 hours and T=4 hours.

TABLE 7
Stability study results for GD2-SADA formulation:

| Analytical | 0.05 mg/ml | | 10 mg/ml | |
|---|---|---|---|---|
| method | T = 0 hours | T = 4 hours | T = 0 hours | T = 4 hours |
| Visible Particles | Essentially free | Essentially free | Essentially free | Essentially free |
| | of visible | of visible | of visible | of visible |
| | particles and | particles and | particles and | particles and |
| | bubbles | bubbles | bubbles | bubbles |
| Subvisible | 5 Particles/mL | 3 Particles/mL | 4 Particles/mL | 4 Particles/mL |
| Particles ≥ 25 μm | | | | |
| Subvisible | 29 Particles/ml | 29 Particles/ml | 52 Particles/ml | 22 Particles/ml |
| Particles ≥ 10 μm | | | | |
| Relative potency | Comparable to | Comparable to | Comparable to | Comparable to |
| (by GD2 SPR) | reference | reference | reference | reference |
| | standard with | standard with | standard with | standard with |
| | (relative) | (relative) | (relative) | (relative) |
| | potency | potency | potency | potency |
| | between 50 | between 50 | between 50 | between 50 |
| | to 150%^{b} | to 150% | to 150% | to 150% |
| A280 (content | 0.06 mg/ml | 0.056 mg/mL | 9.89 mg/mL | 9.89 mg/mL |
| protein) | 113% Recovery | 106% Recovery | 99% Recovery | 99% Recovery |
| Non-reduced SDS | 20.3% | 20.3% | 20.7% | 20.7% |
| cGE | | | | |
| Dimer | | | | |
| Non-reduced SDS | 72.5% | 72.5% | 71.9% | 71.9% |
| cGE | | | | |
| Monomer | | | | |
| Non-reduced SDS | 92.8% | 92.7% | 92.6% | 92.6% |
| cGE | | | | |
| Monomer + | | | | |
| Dimer | | | | |
| Relative potency | 106%^{b} | 87% | 101% | 101% |
| (by GD2 SPR) | | | | |
| Relative potency | 95% | 102% | 104% | 106% |
| (by DOTA SPR) | | | | |
| Non-reduced | 6.4% | 6.5% | 6.7 | 6.7% |
| SDS-cGE HMWS | | | | |
| Non-reduced | 0.8% | 0.8% | 0.7 | 0.7% |
| SDS-cGE LMWS | | | | |
| Reduced SDS-cGE | 1.5% | 1.4% | 1.4% | 1.5% |
| HMWS | | | | |
| Reduced SDS-cGE | 3.3% | 3.2% | 3.4% | 3.4% |
| LMWS | | | | |
| Reduced SDS-cGE | 95.2% | 95.4% | 95.1% | 95.1% |
| Purity | | | | |
| SE-HPLC HMWS | 4.4% | 4.9% | 4.9% | 5.3% |
| SE-HPLC LMWS 1 | 0.7% | 0.6% | 0.7% | 0.7% |
| SE-HPLC | 94.5% | 94.0% | 93.9% | 93.5% |
| Tetramer | | | | |
| SE-HPLC other | 0.5% | 0.6% | 0.5% | 0.5% |
| LMWS | | | | |
| Osmolality | 288 mOsm/kg | 288 mOsm/kg | 337 mOsm/kg | 338 mOsm/kg |
| pH | 5.5 | 5.8 | 5.5 | 5.5 |

Protein recovery was 99 and 106% for the tested concentrations. The recovery was calculated as the percentage difference between the observed concentrations and expected theoretical concentration.

To assess the potential effect of surface adherence to the infusion materials at worst-case conditions, the lowest dose concentration (0.05 mg/mL) was prepared using precision pipettes to minimize variation from the preparation procedure, which would not normally affect the dose administered in the clinic where the entire volume was infused. This experiment identified a protein recovery of 106%, indicating that GD2-SADA surface adherence to the administration materials was negligible.

In conclusion, the formulation used was evaluated to be stable within the tested concentration range of 0.05 mg/ml to 10 mg/mL, as well as during storage and handling of the GD2-SADA for up to 4 hours at room temperature.

### Example 7: Stability to Support Shelf Life

The stability results of the supportive shelf life study are summarized in Table 8 for the long-term stability study (5±3° C.) and in Table 9 for accelerated conditions (25±2° C.).

The long-term stability study shows that GD2-SADA is stable in tetramer form (>94%) at 2-8° C. for at least 9 months in formulations containing 20 mM sodium acetate buffer, 275 mM sucrose and 0.2 g/L polysorbate 20 at pH of 5.5. In addition, the accelerated stability data shows that GD2-SADA is stable in tetramer form (>94%) at 25° C. for at least 3 months.

In addition, the stability data shows that GD2-SADA is stable in current formulation regarding potency, purity and impurity.

TABLE 8
Long-term stability data for the GD2-SADA formulation (stored at 5 ± 3° C.)

| | | 0 months | | | |
|---|---|---|---|---|---|
| Test method | Unit | (M) | 3M | 6M | 9M |
| Degree of | N/A | Slightly | Slightly | Slightly | Slightly |
| opalescence, | | opalescent | opalescent | opalescent | opalescent |
| clarity | | | | | |
| Clarity | NTU | 5 | 6 | 6 | 6 |
| | N/A | <reference | =reference | =reference | =reference |
| | | suspension II | suspension II | suspension II | suspension II |
| Degree of | BY-scale | <BY6 | <BY6 | <BY6 | <BY6 |
| colouration | | | | | |
| of liquids | | | | | |
| Visible | N/A | Essentially | Not tested | Essentially | Not tested |
| particles | | free of visible | | free of visible | |
| | | particles | | particles | |
| A280 | mg/ml | 14.5 | 14.7 | 14.7 | 14.7 |
| (content | | | | | |
| protein) | | | | | |
| Non-reduced | % | 71.4 | 72.6 | 71.9 | 71.4 |
| SDS-cGE | monomer | | | | |
| | % dimer | 20.8 | 20.2 | 20.7 | 20.9 |
| | % | 92.3 | 92.8 | 92.6 | 92.4 |
| | Monomer + | | | | |
| | % dimer | | | | |
| Relative | % | 99 | 106 | 106 | 111 |
| potency SPR | | | | | |
| (DOTA) | | | | | |
| Relative | % | 112 | 108 | 99 | 99 |
| potency SPR | | | | | |
| (GD2) | | | | | |
| Reduced | % purity | 94.9 | 95.8 | 95.0 | 94.7 |
| SDS-cGE | % LMW | 3.9 | 3.1 | 3.7 | 4.3 |
| | species | | | | |
| | % HMW | 1.2 | 1.1 | 1.2 | 1.1 |
| | species | | | | |
| Non-reduced | % HMW | 6.9 | 6.5 | 6.7 | 6.8 |
| | species | | | | |
| SDS-cGE | % LMW | 0.9 | 0.7 | 0.7 | 0.8 |
| | species | | | | |
| SE-HPLC | % | 94.7 | 94.4 | 95.4 | 94.5 |
| | Tetramer | | | | |
| | % HMW | 4.1 | 4.4 | 3.2 | 4.2 |
| | species | | | | |
| | % LMW | 0.6 | 0.7 | 0.8 | 0.7 |
| | species 1 | | | | |
| | % other | 0.6 | 0.5 | 0.6 | 0.6 |
| | LMWs | | | | |
| Bioburden | cfu/10 mL | 0 | Not tested | Not tested | Not tested |
| Osmolality | mOsmol/ | 362 | Not tested | 367 | Not tested |
| | kg | | | | |
| pH | N/A | 5.5 | 5.5 | 5.5 | 5.5 |

TABLE 9
Accelerated stability data for the GD2-SADA formulation (stored at 25 ± 2° C.)

| | | 0 months | | | | | |
|---|---|---|---|---|---|---|---|
| Test method | Unit | (M) | | 1M | 2M | 3M | |
| Degree of | N/A | Reference | | Reference | Reference | Reference | |
| opalescence, | | suspension II | | suspension II | suspension II | suspension II | |
| clarity | | | | | | | |
| Clarity | NTU | 6 | | 6 | 6 | 6 | |
| | N/A | slightly | | slightly | slightly | slightly | |
| | | opalescent | | opalescent | opalescent | opalescent | |
| Degree of | BY-scale | <BY6 | | <BY5 | <BY6 | <BY6 | |
| colouration | | | | | | | |
| of liquids | | | | | | | |
| Container | N/A | no dye | | Not tested | Not tested | Not tested | |
| and closure | | ingress | | | | | |
| Visible | N/A | Essentially | | Not tested | Essentially | Essentially | |
| particles | | free ofvisible | | | free of | free of | |
| | | particles | | | visible | visible | |
| | | | | | particles | particles | |
| Subvisible | N/A | ≥2 μm: | 3494 | Not tested | Not tested | ≥2 μm: | 3737 |
| particles | | ≥5 μm: | 413 | | | ≥5 μm: | 393 |
| (SvP) | | ≥10 μm: | Pass | | | ≥10 μm: | Pass |
| | | ≥25 μm: | Pass | | | ≥25 μm: | Pass |

| | | | | | |
|---|---|---|---|---|---|
| A280 | mg/mL | 14.8 | 14.8 | 14.8 | 14.8 |
| (content | | | | | |
| protein) | | | | | |
| Non-reduced | % | 72.9 | 72.2 | 71.6 | 71.4 |
| SDS-cGE | monomer | | | | |
| | % dimer | 20.2 | 20.6 | 20.3 | 20.2 |
| | % | 93.1 | 92.8 | 91.9 | 91.6 |
| | Monomer + | | | | |
| | % dimer | | | | |
| Relative | % | 102 | 105 | 103 | 112 |
| potency SPR | | | | | |
| (DOTA) | | | | | |
| Relative | % | 100 | 97 | 96 | 94 |
| potency SPR | | | | | |
| (GD2) | | | | | |
| Reduced | % purity | 96.5 | 95.0 | 91.2 | 90.0 |
| SDS-cGE | % LMW | 2.5 | 3.9 | 6.3 | 8.1 |
| | species | | | | |
| | % HMW | 1.0 | 1.1 | 2.5 | 1.9 |
| | species | | | | |
| Non-reduced | % HMW | 6.2 | 6.5 | 6.1 | 6.0 |
| SDS-cGE | species | | | | |
| | % LMW | 0.7 | 0.7 | 0.8 | 0.9 |
| | species | | | | |
| SE-HPLC | % | 95.1 | 95.6 | 94.7 | 94.4 |
| | Tetramer | | | | |
| | % HMW | 4.1 | 3.0 | 3.6 | 3.8 |
| | species | | | | |
| | % LMW | 0.5 | 0.9 | 1.2 | 1.3 |
| | species 1 | | | | |
| | % other | 0.3 | 0.5 | 0.5 | 0.5 |
| | LMWs | | | | |
| Osmolality | mOsmol/ | 368 | Not tested | Not tested | Not tested |
| | kg | | | | |
| pH | N/A | 5.5 | 5.5 | 5.5 | 5.5 |

### Example 8. Thermal Stability of Additional SADA-Conjugates

Sample solution were centrifuged for 1 h at 20,000×g, 4° C. in a tabletop centrifuge. The supernatant was buffer exchanged into stock buffer (Histidine and Acetate with and without 150 mM NaCl) and samples were further diluted to 10 μM. Sucrose was spiked in for all conditions with target sucrose concentration. Each sample was measured as duplicates by nanoDSF (Ratio 350/330 nm for protein unfolding Tm). There are five molecules included in this study. These molecules have the same DOTA binding and P53 sequence, but with different antigen binding sites, such as GD2, CD38, B7H3, CD20 and GPA33.

As shown in table 10 and 11, 150 mM NaCl has a negative impact on thermal stability of all investigated SADA molecules, as indicated by a decreased Tm value, compared with buffer groups (pH5.5 acetate and pH6.0 histidine). In addition, 275 mM sucrose has a positive impact on thermal stability of all investigated SADA molecules, as indicated by an increased Tm value, compared with buffer groups (pH5.5 acetate and pH6.0 histidine). This salt destabilizing and sugar stabilizing effect is universal on all investigated SADA molecules, even though the antigen binding sites are different. Therefore, we conclude the salt destabilizing and sugar stabilizing effect is mainly driven by DOTA binding and P53 part.

TABLE 10
Thermal stability (Tm) overview of tested SADA
molecules in pH 5.5 acetate buffer W/O salt and sugar

| | Acetate buffer | Acetate + 150 mM | Acetate + 275 mM |
|---|---|---|---|
| | (pH 5.5) | NaCl (pH 5.5) | Sucrose (pH 5.5) |
| CD38-SADA(Tm, ° C.) | 60.41 | 59.92 | 61.27 |
| B7H3-SADA(Tm, ° C.) | 53.78 | 52.86 | 55.05 |
| CD20-SADA (Tm₁, ° C.) | 55.85 | 54.67 | 57.2 |
| CD20-SADA (Tm₂, ° C.) | 71.2 | 68.55 | 72.37 |
| GD2-SADA(Tm, ° C.) | 60.74 | 60.42 | 61.91 |
| GPA33-SADA (Tm₁, ° C.) | 59.87 | 60.62 | 61.02 |
| GPA33-SADA (Tm₂, ° C.) | 70.10 | 65.43 | 71.35 |

TABLE 11
Thermal stability (Tm) overview of tested SADA
molecules in pH 6.0 histidine buffer W/O salt and sugar

| | Histidine buffer | Histidine + 150 mM | Histidine + 275 mM |
|---|---|---|---|
| | (pH 6.0) | NaCl (pH 6.0) | Sucrose (pH 6.0) |
| CD38-SADA(Tm,° C.) | 60.68 | 60.09 | 61.10 |
| B7H3-SADA(Tm,° C.) | 53.93 | 53.69 | 55.07 |
| CD20-SADA (Tm₁, ° C.) | 56.05 | 54.9 | 57.3 |
| CD20-SADA (Tm₂, ° C.) | 71.7 | 68.56 | 72.35 |
| GD2-SADA(Tm,° C.) | 60.8 | 60.5 | 62.04 |
| GPA33-SADA (Tm₁, ° C.) | 59.93 | 60.19 | 61.21 |
| GPA33-SADA (Tm₂, ° C.) | 70.26 | 65.71 | 71.53 |

### Example 9. GD2-SADA Tetramer has Better PK Profile and Thus Better Tumor Update as Well as the Tumor Killing Effect

To better evaluate tetrameric role, we compared GD2-SADA drug candidate to an obligate monomeric version, termed (P53−/−) GD2-SADA, where the entire SADA domain was eliminated, resulting in a final protein size of approximately 54 kDa.

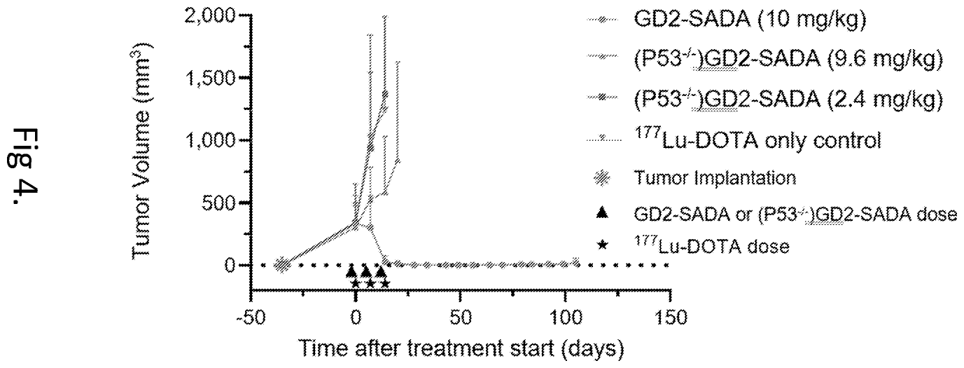

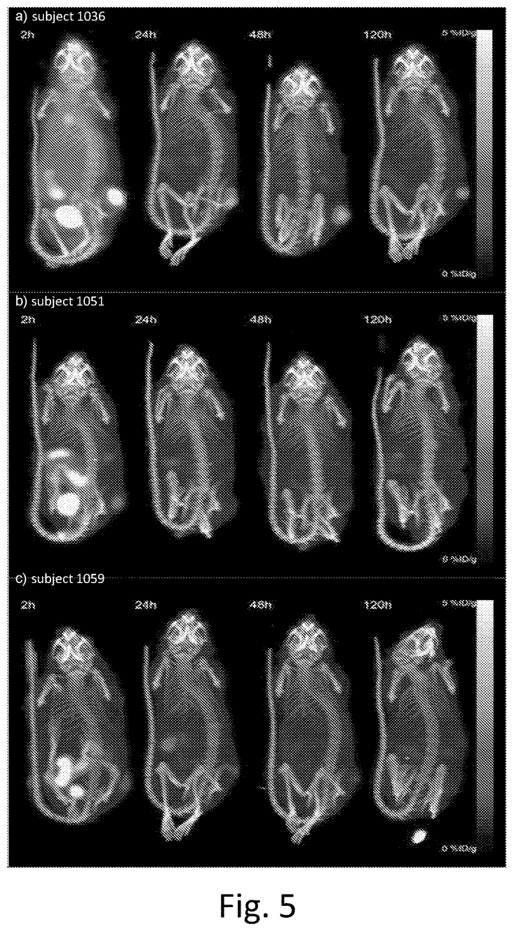

GD2-SADA and (P53−/−) GD2-SADA were compared regarding plasma pharmacokinetics and anti-tumor efficacy. In summary, the result demonstrates GD2-SADA tetramer, altering the plasma exposure profile (Table 12), and improving the therapeutic efficacy of GD2-SADA (FIG. 4), compared to GD2-SADA, (P53−/−) GD2-SADA in monomer form. This was also supported by SPECT/CT imaging (FIG. 5) which demonstrated substantially higher tumor binding and persistence for GD2-SADA compared to (P53−/−) GD2-SADA monomer. Therefore, it is important to keep GD2-SADA in tetramer form by current claimed composition.

TABLE 12
(P53^{-/-})GD2-SADA pharmacokinetic data summary (n = 6)

| | Dose level | Half-life | | Cₘₐₓ | AUC_{0.5-72} |
|---|---|---|---|---|---|
| Study groups | (per mouse) | (hr) | Tₘₐₓ | (% ID/g) | (h × % ID/g) |
| (P53^{-/-})GD2-SADA | 9.6 mg/kg | 7.8 | 0.5 h | 17.2 ± 3.4 | 125.5 ± 3.7 |
| (high dose) | (240 μg) | | | | |
| (P53^{-/-})GD2-SADA | 2.4 mg/kg | 6.9 | 0.5 h | 18.5 ± 1.4 | 138.8 ± 2.6 |
| (low dose) | (60 μg) | | | | |
| GD2-SADA | 10 mg/kg | 8.8 | 0.5 h | 45.6 ± 19.0 | 330.3 ± 21.5 |
| (reference dose) | (250 μg) | | | | |
| GD2-SADA | 2.5 mg/kg | 5.8 | 0.5 h | 41.4 ± 0.6 | 362.6 ± 6.4 |
| (low dose) | (62.5 μg) | | | | |

### Example 10. Phase I Trial of GD2-SADA: ¹⁷⁷Lu-DOTA Drug Complex in Patients with Recurrent or Refractory Metastatic Solid Tumors Known to Express GD2, Including Small Cell Lung Cancer, Sarcoma and Malignant Melanoma

The trial will be divided in 3 separate parts:
A. GD2-SADA dose escalation to optimize the safe tumor-targeting protein component (GD2-SADA) dose and dosing interval between GD2-SADA and ¹⁷⁷Lu-DOTA administrations.
B. ¹⁷⁷Lu-DOTA dose escalation to establish optimal, safe payload administration for therapy.
C. Repeated dosing for assessment of cumulative toxicity signals and safety profile following repeated dosing and determination of the recommended phase 2 dose.

The patient population will consist of adult and adolescent patients with recurrent or refractory metastatic solid tumors known to express GD2, including Small Cell Lung Cancer (SCLC), Sarcoma and Malignant Melanoma.

The aim of the trial is to establish a safe dosing schedule.

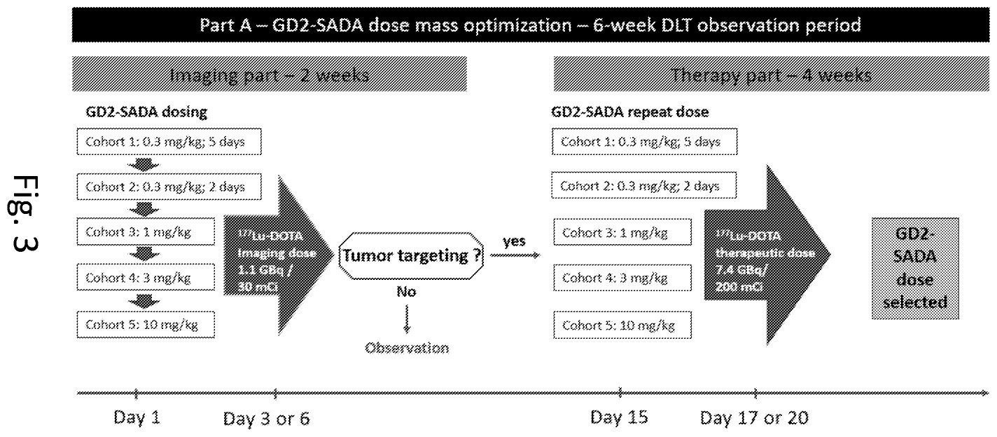

The trial design of part A can be seen in FIG. 3 and table 13 below.

TABLE 13
Part A treatment schedule:

| | Imaging Part | | | | Therapy Part | | | |
|---|---|---|---|---|---|---|---|---|
| | | | | ¹⁷⁷Lu- | | | | ¹⁷⁷Lu- |
| | GD2-SADA | | Interval | DOTA | GD2-SADA | | Interval | DOTA |
| | dose | | (h) | dose | dose | | (h) | dose |
| Cohort 1 | 0.3 | mg/kg | 120 | 1.1 GBq/ | 0.3 | mg/kg | 120 | 7.4 GBq/ |
| | | | | 30 mCi | | | | 30 mCi |
| Cohort 2 | 0.3 | mg/kg | 48 | 1.1 GBq/ | 0.3 | mg/kg | 48 | 7.4 GBq/ |
| | | | | 30 mCi | | | | 30 mCi |
| Cohort 3 | 1 | mg/kg | 48 or 120 | 1.1 GBq/ | 1 | mg/kg | 48 or 120 | 7.4 GBq/ |
| | | | | 30 mCi | | | | 30 mCi |
| Cohort 4 | 3 | mg/kg | 48 or 120 | 1.1 GBq/ | 3 | mg/kg | 48 or 120 | 7.4 GBq/ |
| | | | | 30 mCi | | | | 30 mCi |
| Cohort 5 | 10 | mg/kg | 48 or 120 | 1.1 GBq/ | 10 | mg/kg | 48 or 120 | 7.4 GBq/ |
| | | | | 30 mCi | | | | 30 mCi |

Cohort 1: Patients will be administered an intravenous infusion of GD2-SADA on Day 1 followed by an intravenous infusion of ¹⁷⁷Lu-DOTA imaging dose on Day 6. On Day 15 a repeated dose of GD2-SADA will be administered followed by a therapy dose of ¹⁷⁷Lu-DOTA on day 20.
Cohort 2: Patients will be administered an intravenous infusion of GD2-SADA on Day 1 followed by an intravenous infusion of ¹⁷⁷Lu-DOTA imaging dose on Day 3. On Day 15 a repeated dose of GD2-SADA will be administered followed by a therapy dose of ¹⁷⁷Lu-DOTA on day 17.
Cohort 3-5: Patients will be dosed on the same days as either Cohort 1 or Cohort 2, depending on the dosing interval between GD2-SADA and ¹⁷⁷Lu-DOTA selected after analysis of data from the first two cohorts.

In Part A dosimetry including tumor absorbed dose and whole body, selected organ absorbed doses and blood dosimetry will be assessed.

Part A will consist of a 6-week DLT observation period and a follow-up period lasting up to 24 weeks after first treatment.

Assuming a 48-hour interval between GD2-SADA and ¹⁷⁷Lu-DOTA is selected in Part A, patients will be administered an intravenous infusion of GD2-SADA on Day 1 followed by an intravenous infusion of ¹⁷⁷Lu-DOTA on day 3. On day 15, a repeated dose of GD2-SADA will be administered followed by a therapy dose of ¹⁷⁷Lu-DOTA on day 17.

On Day 43 (GD2-SADA) and Day 45 (¹⁷⁷Lu-DOTA) the second treatment cycle will be administered.

The trial design of Part B can be seen in table 14 below.

TABLE 14
Part B Treatment schedule

| | Imaging Part | | | Therapy Part | | |
|---|---|---|---|---|---|---|
| | | | ¹⁷⁷Lu- | | | ¹⁷⁷Lu- |
| | GD2-SADA | | DOTA | GD2-SADA | | DOTA |
| | dose | Interval | dose | dose (Day | Interval | dose (Day |
| | (Day 1) | (h) | (Day 3) | 15 & 43) | (h) | 17 and 45) |
| Cohort 1 | Dose | 48 | 1.1 GBq/ | Dose | 48 | 14.8 GBq/ |
| | selected in | | 30 mCi | selected in | | 400 mCi |
| | Part A | | | Part A | | |
| Cohort 2 | Dose | 48 | 1.1 GBq/ | Dose | 48 | 22.2 GBq/ |
| | selected in | | 30 mCi | selected in | | 600 mCi |
| | Part A | | | Part A | | |
| Cohort 3 | Dose | 48 | 1.1 GBq/ | Dose | 48 | 27.8 GBq/ |
| | selected in | | 30 mCi | selected in | | 750 mCi |
| | Part A | | | Part A | | |

Part B will consist of a 6-week DLT observation period and a follow-up period lasting up to 24 weeks after first treatment.

The treatment scheduled in Part C assumes a 48-hour interval between GD2-SADA and ¹⁷⁷Lu-DOTA has been selected in Part A. Patients will be administered an intravenous infusion of GD2-SADA on Day 1 followed by an intravenous infusion of ¹⁷⁷Lu-DOTA imaging dose on Day 3.

On Day 15, a repeated dose of GD2-SADA will be administered followed by a therapy dose of ¹⁷⁷Lu-DOTA on Day 17.

First treatment cycle (Imaging Part followed by Therapy Part) is planned to have a duration of 6 weeks, and subsequent cycles (cycle 2-5) are planned to be 4 weeks (or when recovery from radiation toxicities incurred—a maximum delay of 8 weeks was permitted within this protocol) with GD2-SADA dosing on Day 1 and ¹⁷⁷Lu-DOTA dosing on Day 3 of each cycle. See Table 15 for Treatment schedule.

TABLE 15
Part C treatment Schedule:

| | | | | Treatment cycle 1-5 | | |
|---|---|---|---|---|---|---|
| | | | | GD2-SADA | | ¹⁷⁷Lu-DOTA |
| | Imaging Part | | | dose (Day 15 | | dose (Day 17 |
| | GD2-SADA | | ¹⁷⁷Lu-DOTA | of cycle 1 | | of cycle 1 |
| | dose | Interval | dose | and Day 1 of | Interval | and Day 3 of |
| | (Day 1) | (h) | (Day 3) | cycle 2-5) | (h) | cycle 2-5) |
| All | Dose | 48 | 1.1 GBq/ | Dose | 48 | Dose |
| patients | selected in | | 30 mCi | selected in | | selected in |
| | Part A | | | Part A | | Part B |

Part C will consist of a 6-week treatment cycle (Cycle 1) followed by up to 4 treatment cycles (Cycles 2-5) with a duration of 4 weeks each and a follow-up period lasting up to 52 weeks after first treatment.

Pre-medication including analgesic is mandatory and introduced based on experience from anti-GD2 IgG-based monoclonal antibodies. Administration scheme can be seen below in Table 16.

TABLE 16
GD2-SADA Pre- and post-medication

| Day | Medication |
|---|---|
| −1 | Analgesic: Gabapentin 300 mg × 1 |
| 0 | Analgesic: Gabapentin 300 mg × 2 |
| 1 (0.5-1 h | Analgesic: Morphine sulfate (50 mcg/kg) IV |
| prior to | 30 min prior to |
| SADA | initiation of GD2 SADA infusion |
| infusion) | Anti-inflammatory: dexamethasone 10 mg |
| | IV (or equivalent) |
| | Anti-nausea: Ondansetron (Zofran), 8 mg |
| | PO or equivalent. |
| | Antipyretic: Acetaminophen (paracetamol) |
| | 1 g PO (or equivalent), |
| 2 | Analgesic: Gabapentin 300 mg × 3 |
| | Anti-inflammatory: dexamethasone 10 mg |
| | PO (or equivalent) |
| 3 | Analgesic: Gabapentin 300 mg × 3 |

Sequences:

SEQ ID NO. 1: KASQSVSNDVT
SEQ ID NO. 2: SASNRYS
SEQ ID NO. 3: QQDYSS
SEQ ID NO. 4: NYGVH
SEQ ID NO. 5: VIWAGGITNYNSAFMS
SEQ ID NO. 6: RGGHYGYALDY
SEQ ID NO. 7:
EIVMTQTPATLSVSAGERVTITCKASQSVSNDVTWYQQKPGQAPRLLIYSASNRYSGVPARFSGSGYGTE
FTFTISSVQSEDFAVYFCQQDYSSFGCGTKLEIKRGGGGSGGGGSGGGGSGGGGSGGGGSGGGGSQVQ
LVESGPGVVQPGRSLRISCAVSGFSVTNYGVHWVRQPPGKCLEWLGVIWAGGITNYNSAFMSRLTISKD
NSKNTVYLQMNSLRAEDTAMYYCASRGGHYGYALDYWGQGTLVTVSS
SEQ ID NO. 8:
EIVMTQTPATLSVSAGERVTITCKASQSVSNDVTWYQQKPGQAPRLLIYSASNRYSGVPARFSGSGYGTE
FTFTISSVQSEDFAVYFCQQDYSSFGCGTKLEIKR
SEQ ID NO. 9:
QVQLVESGPGVVQPGRSLRISCAVSGFSVTNYGVHWVRQPPGKCLEWLGVIWAGGITNYNSAFMSRLTI
SKDNSKNTVYLQMNSLRAEDTAMYYCASRGGHYGYALDYWGQGTLVTVSS
SEQ ID NO. 10:
QAVVTQEPSLTVSPGGTVTLTCGSSTGAVTASNYANWVQQKPGQCPRGLIGGHNNRPPGVPARFSGSL
LGGKAALTLLGAQPEDEAEYYCALWYSDHWVIGGGTKLTVLG
SEQ ID NO. 11:
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGCGTLVTVSS
SEQ ID NO. 12: KPLDGEYFTLQIRGRERFEMFRELNEALELKDAQAGKEP
SEQ ID NO. 13: RSPDDELLYLPVRGRETYEMLLKIKESLELMQYLPQHTIETYRQQQQQQHQHLLQK
SEQ ID NO. 14: RHGDEDTYYLQVRGRENFEILMKLKESLELMELVPQPLVDSYRQQQQLLQRP
SEQ ID NO. 15: QAIKKELTQIKQKVDSLLENLEKIEKE
SEQ ID NO. 16: STRRILGLAIESQDAGIKTITMLDEQKEQLNRIEEGLDQINKDMRETEKTLTEL
SEQ ID NO. 17:
MCGAPSATQPATAETQHIADQVRSQLEEKENKKFPVFKAVSFKSQVVAGTNYFIKVHVGDEDFVHLRVF
QSLPHENKPLTLSNYQTNKAKHDELTYF
SEQ ID NO. 18: DEISMMGRVVKVEKQVQSIEHKLDLLLGFY
SEQ ID NO. 19:
TVAEAKRQAAEDALAVINQQEDSSESCWNCGRKASETCSGCNTARYCGSFCQHKDWEKHH
SEQ ID NO. 20: GGGGS
SEQ ID NO. 21: TPLGDTTHT
SEQ ID NO. 22:
EIVMTQTPATLSVSAGERVTITCKASQSVSNDVTWYQQKPGQAPRLLIYSASNRYSGVPARFSGSGYGTE
FTFTISSVQSEDFAVYFCQQDYSSFGCGTKLEIKRGGGGSGGGGSGGGGSGGGGSGGGGSGGGGSQVQ
LVESGPGVVQPGRSLRISCAVSGFSVTNYGVHWVRQPPGKCLEWLGVIWAGGITNYNSAFMSRLTISKD
NSKNTVYLQMNSLRAEDTAMYYCASRGGHYGYALDYWGQGTLVTVSSGGGGSGGGGSGGGGSGGG
GSHVQLVESGGGLVQPGGSLRLSCAASGFSLTDYGVHWVRQAPGKGLEWLGVIWSGGGTAYNTALISR
FTISRDNSKNTLYLQMNSLRAEDTAVYYCARRGSYPYNYFDAWGCGTLVTVSSGGGGSGGGGSGGGGS
GGGGSGGGGSGGGGSQAVVTQEPSLTVSPGGTVTLTCGSSTGAVTASNYANWVQQKPGQCPRGLIGG
HNNRPPGVPARFSGSLLGGKAALTLLGAQPEDEAEYYCALWYSDHWVIGGGTKLTVLGTPLGDTTHTSG
KPLDGEYFTLQIRGRERFEMFRELNEALELKDAQAGKEPGGSGGA
SEQ ID NO. 23: TGAVTASNY
SEQ ID NO. 24: GHN
SEQ ID NO. 25: ALWYSDHWV
SEQ ID NO. 26: GFSLTDYG
SEQ ID NO. 27: IWSGGGT
SEQ ID NO. 28: ARRGSYPYNYFDA
SEQ ID NO. 29: EDIYNR
SEQ ID NO. 30: GAT
SEQ ID NO. 31: QQYWSNPYT
SEQ ID NO. 32: GFSLTSYG
SEQ ID NO. 33: MWRGGST
SEQ ID NO. 34: AKSMITTGFVMDS
SEQ ID NO. 35:
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSSGGGGSGGGGSGGGGSGG
GGSGGGGSGGGGSDIQLTQSPSSLSASVGDRVTITCKASEDIYNRLTWYQQKPGKAPKLLISGATSLETGV
PSRFSGSGSGKDYTFTISSLQPEDFATYYCQQYWSNPYTFGQGTKLEIK
SEQ ID NO. 36:
DIQLTQSPSSLSASVGDRVTITCKASEDIYNRLTWYQQKPGKAPKLLISGATSLETGVPSRFSGSGSGKDYTF
TISSLQPEDFATYYCQQYWSNPYTFGQGTKLEIK
SEQ ID NO. 37:
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSS
SEQ ID NO. 38:
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSSGGGGSGGGGSGGGGSGG
GGSGGGGSGGGGSDIQLTQSPSSLSASVGDRVTITCKASEDIYNRLTWYQQKPGKAPKLLISGATSLETGV
PSRFSGSGSGKDYTFTISSLQPEDFATYYCQQYWSNPYTFGQGTKLEIKGGGGSGGGGSGGGGSGGGGS
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGQGTLVTVSSGGGGSGGGGSGGGGSGG
GGSGGGGSGGGGSQAVVTQEPSLTVSPGGTVTLTCGSSTGAVTASNYANWVQQKPGQAPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAP
SEQ ID NO: 39:
QVQLVQSGAEVKKPGASVKVSCKASGYTFTNYDINWVRQATGQGLEWMGWIFPGDGSTQYNEKFQG
RVTMTTNTSISTAYMELSSLRSEDTAVYYCARQTTATWFAYWGQGTLVTVSSGGGGSGGGGSGGGGSG
GGGSGGGGSGGGGSEIVMTQSPATLSVTPKEKVTITCRASQSISDYLHWYQQKPDQSPKLLIKYASQSISG
VPSRFSGSGSGSDFTLTINSLEAEDAATYYCQNGHSFPLTFGQGTKLEIKGGGGSGGGGSGGGGSGGGGS
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGQGTLVTVSSGGGGSGGGGSGGGGSGG
GGSGGGGSGGGGSQAVVTQEPSLTVSPGGTVTLTCGSSTGAVTASNYANWVQQKPGQAPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAP
SEQ ID NO: 40:
QIVLSQSPAILSASPGEKVTMTCRASSSVSYIHWFQQKPGSSPKPWIYATSNLASGVPVRFSGSGSGTSYSL
TISRVEAEDAATYYCQQWTSNPPTFGGGTKLEIKGGGGSGGGGSGGGGSGGGGSGGGGSGGGGSQV
QLQQPGAELVKPGASVKMSCKASGYTFTSYNMHWVKQTPGRGLEWIGAIYPGNGDTSYNQKFKGKAT
LTADKSSSTAYMQLSSLTSEDSAVYYCARSTYYGGDWYFNVWGAGTTVTVSAGGGGSGGGGSGGGGS
GGGGSHVQLVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGQGTLVTVSSGGGGSGGGGSGG
GGSGGGGSGGGGSGGGGSQAVVTQEPSLTVSPGGTVTLTCGSSTGAVTASNYANWVQQKPGQAPRG
LIGGHNNRPPGVPARFSGSLLGGKAALTLLGAQPEDEAEYYCALWYSDHWVIGGGTKLTVLGTPLGDTT
HTSGKPLDGEYFTLQIRGRERFEMFRELNEALELKDAQAGKEPGGSGGA
SEQ ID NO: 41:
EVQLVESGGGLVKPGGSLRLSCAASGFAFSTYDMSWVRQAPGKCLEWVSTISSGGSYTYYADSVKGRFTI
SRDNAKNSLYLQMNSLRAEDTAVYYCAPTTVVPFAYWGQGTLVTVSAGGGGSGGGGSGGGGSGGGGS
GGGGSGGGGSDIQMTQSPSSLSASVGDRVTITCKASQNVRTVVAWYQQKPGKAPKTLIYLASNRHTGV
PSRFSGSGSGTEFTLTISNLQPEDFATYYCLQHWSYPLTFGCGTKLEVKRGGGGSGGGGSGGGGSGGGG
SHVQLVESGGGLVQPGGSLRLSCAASGFSLTDYGVHWVRQAPGKGLEWLGVIWSGGGTAYNTALISRFT
ISRDNSKNTLYLQMNSLRAEDTAVYYCARRGSYPYNYFDAWGCGTLVTVSSGGGGSGGGGSGGGGSGG
GGSGGGGSGGGGSQAVVTQEPSLTVSPGGTVTLTCGSSTGAVTASNYANWVQQKPGQCPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAP

1. An aqueous composition comprising
a. A SADA-complex, comprising a SADA domain, a first binding site, a second binding site, in an amount of 5-50 g/L;
b. A buffer-system;
c. One or more stabilizing agent(s); and
d. One or more surfactant(s).

2. The composition of claim 1, where in the pH is in the range of 5-6.

3. The composition according to claim 1 or 2, wherein the ionic strength is in the range of 5-150 mM.

4. The composition according to any of the preceding claims, wherein the ionic strength is in a range selected among 5-150 mM, 15-135 mM, 20-120 mM and 25-100 mM.

5. The composition according to any of the preceding claims, comprising a SADA-complex in an amount selected among 5-50 g/L, 6.25-45 g/L, 7.5-40 g/L, 9.75-35 g/L, 10-30 g/L, 11.25-25 g/L, 12.5-20 g/L, 13.75-17.5 g/L and preferably 15 g/L.

6. The composition according to any of the preceding claims, wherein the first binding site is capable of binding to a tumor antigen.

7. The composition according to claim 6, wherein the first binding site is capable of binding to GD2, B7-H3, CD20, GPA33 or CD38.

8. The composition according to claim 7, wherein the first binding site is capable of binding GD2, comprises the CDR sequences shown in SEQ ID NO: 1-6, and has at least 90%, 95%, 96%, 97%, 98% or preferably at least 99% sequence identity to SEQ ID NO: 7.

9. The composition according to claim 7, wherein the first binding site is capable of binding GD2, and comprises:
a. a polypeptide comprising a sequence with at least 90%, 95%, 96%, 97%, 98% or preferably at least 99% sequence identity to SEQ ID NO: 8, and
b. a polypeptide comprising a sequence with at least 90%, 95%, 96%, 97%, 98% or preferably at least 99% sequence identity to SEQ ID NO: 9.

10. The composition according to claim 7, wherein the first binding site is capable of binding to CD38, comprises the CDR sequences shown in SEQ ID NO: 29-34 and has at least 90%, 95%, 96%, 97%, 98% or preferably at least 99% sequence identity to SEQ ID NO: 35.

11. The composition according to claim 7, wherein the first binding site is capable of binding CD38 and comprises:
a. a polypeptide comprising a sequence with at least 90%, 95%, 96%, 97%, 98% or preferably at least 99% sequence identity to SEQ ID NO: 36, and
b. a polypeptide comprising a sequence with at least 90%, 95%, 96%, 97%, 98% or preferably at least 99% sequence identity to SEQ ID NO: 37.

12. The composition according to any of the claims 6-11, wherein the second binding site is capable of binding to a chelator, or a chelator complexing a metal ion.

13. The composition according to claim 12, wherein the second binding site is capable of binding to DOTA, Benzyl-DOTA, a compound comprising the DOTA ring system; or is capable of binding DOTA complexing a metal ion, e.g. lutetium.

14. The composition according to claim 13, wherein the second binding site comprises a polypeptide with a sequence comprising the CDR sequences of SEQ ID NO: 23-28.

15. The composition according to claim 14, wherein the second binding site comprises:
a. a polypeptide comprising a sequence with at least 90%, 95%, 96%, 97% 98% or preferably at least 99% sequence identity, to SEQ ID NO: 10, and
b. a polypeptide comprising a sequence with at least 90%, 95%, 96%, 97% 98% or preferably at least 99% sequence identity, to SEQ ID NO: 11.

16. The composition according to any of the preceding claims, wherein the SADA-domain comprises a polypeptide with a sequence according to SEQ ID NO: 12-19, or a sequence that differs from one of SEQ ID NO: 12-19 by 1, 2, 3, 4, 5, 6, 7, 8, 9, or 10 substitutions.

17. The composition according to claim 16, where the SADA-domain comprises a polypeptide with the sequence of amino acids 6-36 of SEQ ID NO: 12.

18. The composition according to any of the preceding claims, wherein the SADA complex further comprises one or more linkers.

19. The composition according to claim 18, wherein the SADA complex comprises one or more linkers with a sequence selected among SEQ ID NO 20 multiplied by an integer between 1-6 and SEQ ID NO: 21.

20. The composition according to any of the preceding claims wherein the SADA complex comprises a sequence according to SEQ ID NO 22, SEQ ID NO: 38, SEQ ID NO: 39, SEQ ID NO: 40 or SEQ ID NO: 41.

21. The composition according to any of the preceding claims, wherein the buffer system comprises an organic acid or an alkali metal salt thereof.

22. The composition according to claim 21, wherein the organic acid is selected among: acetate, citrate, histidine, citrate-histidine, acetate-histidine and succinate.

23. The composition according to claim 21 or 22, wherein the buffer comprises Sodium Acetate.

24. The composition according to any of the preceding claims, comprising a buffer in an amount selected among 5-100 mM, 15-50 mM and preferably 20 mM.

25. The composition according to any of the preceding claims, wherein the one or more stabilizing agent(s) is/are selected among polyols, sugar alcohols and non-reducing sugars.

26. The composition according to claim 25, wherein the stabilizing agent is sucrose.

27. The composition according to any of the preceding claims comprising a stabilizing agent in an amount selected among 200-350 mM, 250-300 mM, and preferably 275 mM.

28. The composition according to any of the preceding claims, wherein the one or more surfactant(s) is/are selected among nonionic surfactants such as Polyethylene glycol sorbitan monolaurate, Poly(ethylene glycol)-block-poly(propylene glycol)-block-poly(ethylene glycol) or Polyethylene glycol sorbitan monooleate.

29. The composition according to claim 28, wherein the surfactant is Polyethylene glycol sorbitan monolaurate.

30. The composition according to any of the preceding claims comprising a surfactant in an amount selected among 0.1-0.3 g/L, 0.15-0.25 g/l, and preferably 0.20 g/L.

31. The composition according to any of the preceding claims, wherein the pH is selected among 5-6, 5.2-5.8, 5.4-5.6 and preferably 5.5.

32. The composition according to any of the previous claims, further comprising an antioxidant.

33. The composition according to claim 32, wherein the antioxidant is Methionine.

34. The composition according to claim 32 or 33, comprising an antioxidant in an amount selected among 5-15 mM, 8-12 mM and preferably 10 mM.

35. The composition according to any of the preceding claims, wherein the composition does not comprise NaCl or comprises NaCl in a low concentration.

36. The composition of claim 35, where the concentration of NaCl is below 50 mM.

37. The composition according to any of the preceding claims, comprising
a. a SADA-complex comprising or consisting of the amino acid sequence of SEQ ID NO: 22, in an amount of about 15 g/L;
b. Sodium acetate in an amount of about 20 mM;
c. Sucrose in an amount of about 275 mM;
d. Polysorbate 20 in an amount of about 0.2 g/L; and
e. 10 mM methionine;
wherein the pH is 5.5.

38. The composition according to any of the preceding claims, wherein the composition is a pharmaceutical composition.

39. Use of a composition according to any of the previous claims for treating or diagnosing cancer.

40. Use according to claim 40, for treating or diagnosing a cancer expressing GD2, B7-H3, CD20, GPA33 or CD38.

41. Use according to claim 39 or 40, wherein said cancer is selected among neuroblastoma, melanoma, sarcoma, brain tumor or carcinoma.

42. Use according to any of the claims 39-41, wherein said cancer is selected among osteosarcoma, liposarcoma, fibrosarcoma, malignant fibrous histiocytoma, leiomyosarcoma, spindle cell sarcoma, brain tumor, small cell lung cancer, retinoblastoma, HTLV-1 infected T cell leukemia and other GD2 or CD38 positive tumors.

43. Use according to any of the claims 39-42, in a method comprising the steps:
a. Administering a composition according to any of the claims 1-38, to a patient in need of the treatment or diagnosing; and
b. After a period administering a DOTA-compound comprising a radionuclide.

44. Use according to claim 43, wherein the method further comprises administering a clearing agent after step a. and before step b.

45. Use according to claim 43 or 44, wherein the radionuclide is selected among an alpha, beta and positron emitting radionuclide.

46. Use according to claim 45, wherein the radionuclide is selected from the group consisting of ¹⁷⁷Lu, ^{99m}Tc, ⁶⁴Cu, ⁹⁰Y and ⁸⁹Zr.

47. Kit comprising the composition of the claims 1-38, and a DOTA compound.

48. Kit according to claim 47, further comprising a radionuclide.
